(19) United States
(12) Patent Application Publication
(10) Pub. No.: US 2004/0072211 A1
(43) Pub. Date: Apr. 15, 2004

(54) Methods for identifying ligands using waterlogsy NMR

(75) Inventor: Claudio Dalvit, Milan (IT)

Correspondence Address: MUETING, RAASCH & GEBHARDT, P.A., P.O. BOX 581415, MINNEAPOLIS, MN, 55458, US

(73) Assignee: PHARMACIA & UPJOHN COMPANY, Kalamazoo, MI

(21) Appl. No.: 10/454,898

(22) Filed: Jun. 5, 2003

Related U.S. Application Data
(60) Provisional application No. 60/453,504, filed on Mar. 9, 2003.
(60) Provisional application No. 60/386,896, filed on Jun. 5, 2002.

(51) Int. Cl. C12Q 1/68; G01N 33/53

(52) U.S. Cl. 435/6; 435/7.1

(57) ABSTRACT

A method of identifying a ligand to a target molecule that includes: providing a reference compound that interacts with the target molecule; collecting a WaterLOGSY nuclear magnetic resonance spectrum of the reference compound in the presence of the target molecule; providing a test sample comprising at least one test compound; collecting a WaterLOGSY nuclear magnetic resonance spectrum of the reference compound in the presence of the test sample and the target molecule; and comparing the WaterLOGSY spectra to determine if at least one test compound interacts with the target molecule with a binding affinity tighter than that of the reference compound.

This application claims the benefit of U.S. Provisional Application Serial No. 60/386,896 filed Jun. 5, 2002 and U.S. Provisional Application Serial No. 60/453,504, filed Mar. 9, 2003, both of which are incorporated herein by reference in their entireties.

### BACKGROUND OF THE INVENTION

Nuclear magnetic resonance-based (NMR-based) screening has emerged as a potent technique for the identification of small molecules that interact with a protein drug target. Although this methodology suffers from its intrinsic low sensitivity and therefore it requires significantly more protein material than other screening methods, the results obtained with NMR are more reliable. The method is less prone to the type of artifacts observed with other techniques. Recent improvements in cryogenic NMR probe technology enable one to reduce the amount of protein needed for the screening and therefore permit NMR to be competitive with other screening assays.

NMR-based screening can be performed either by monitoring the protein target signals or the ligand signals. Observation of the protein signals provides useful structural information of the ligand-binding mode. In addition, the technique is not restricted by the size of the ligands or by an upper limit in the ligand dissociation binding constant. However, the method requires large amounts of isotope-labelled protein and its application is limited to the observation of small proteins, although relaxation-optimised techniques (TROSY) can extend the molecular sizes amenable to NMR beyond 100 kilodaltons (kDa).

Ligand-observed screening is not limited by the size of the protein and does not require isotope-labelled proteins. Several methods based on the ligand observation have been proposed in the literature. One of these techniques is the WaterLOGSY (Water-Ligand Observed via Gradient SpectroscopY) experiment where the large bulk water magnetization is partially transferred via the protein-ligand complex to the free ligand. Certain methods are limited in their ability to detect strongly binding ligands with slow dissociation rates. In the assumption of a diffusion-limited on-rate of 10⁸ M⁻¹s⁻¹ the upper limit of detection is represented by molecules with dissociation binding constant K_{D} in the 100 nanomolar (nM) range.

Compounds binding tighter to the protein or compounds that have a slow on-rate will not be detected because the residence time of these compounds within the protein is longer than the window of the mixing time (e.g., 1 to 2 seconds) employed in conventional NMR experiments. Thus, what is needed are additional NMR methods that can be used to detect such relatively strong binders, as well as others that are not necessarily such strong binders.

### SUMMARY OF THE INVENTION

The present invention is related to rational drug design. Specifically, the present invention provides a nuclear magnetic resonance (NMR) method of screening for compounds that interact with a target molecule (e.g., typically a protein). The method involves the use of WaterLOGSY (water-ligand observation with gradient spectroscopy) experiments to detect the binding interaction.

Preferably, the present invention is directed to the use of WaterLOGSY in competition binding experiments. Competition binding experiments involve the displacement of a reference compound in the presence of a competing molecule. Preferably, the reference compound interacts with the target molecule with a binding affinity in the micromolar range. Preferably, the test compound interacts with the target molecule with a binding affinity stronger than (i.e., less than) 1 micromolar (e.g., in the nanomolar range). The test compound (i.e., potential ligand) is identified as a ligand if it displaces the reference compound from the target molecule.

In a specific embodiment, the present invention provides a method of identifying a ligand to a target molecule. The method includes: providing a reference compound that interacts with the target molecule; collecting a first WaterLOGSY nuclear magnetic resonance spectrum of the reference compound in the presence of the target molecule; providing a test sample comprising at least one test compound; collecting a second WaterLOGSY nuclear magnetic resonance spectrum of the reference compound in the presence of the test sample and the target molecule; and comparing the first and second WaterLOGSY spectra to determine if at least one test compound interacts with the target molecule by displacing the reference compound. Preferably, the test compound has a binding affinity to the target molecule tighter than that of the reference compound.

In another embodiment, the method optionally further includes: collecting a third WaterLOGSY nuclear magnetic resonance spectrum of the reference compound in the absence of the target molecule; and comparing the WaterLOGSY spectra of the reference compound in the presence of the target molecule, and in the absence of the target molecule, and in the presence of the test sample and target molecule (i.e., first, second, and third spectra) to determine the dissociation constant of the test compound.

Preferably, the step of comparing the WaterLOGSY spectra to determine if at least one test compound interacts with the target molecule involves evaluating at least one reference compound resonance for a change in sign (i.e., by virtue of the opposite sign of their water-ligand nuclear Overhauser effects (NOEs)). Alternatively, this can involve evaluating at least one reference compound resonance for a reduction in signal intensity.

Preferably, the step of identifying the reference compound includes: collecting a WaterLOGSY nuclear magnetic resonance spectrum of a potential reference compound in the absence of the target molecule; collecting a WaterLOGSY nuclear magnetic resonance spectrum of the potential reference compound in the presence of the target molecule; and comparing the WaterLOGSY spectra of the potential reference compound in the presence and the absence of the target molecule to identify whether the potential reference compound interacts with the target molecule.

The present invention could also find useful applications for rapid screening of chemical mixtures (i.e., mixtures of two or more test compounds) such as plant and fungi extracts. Rapid screening techniques typically involve providing a plurality of test samples, each test sample comprising one or more test compound (and often a chemical mixture).

### BRIEF DESCRIPTION OF THE DRAWINGS

FIGS. 1A and 1B. WaterLOGSY signal attenuation of the reference compound as a function of the dissociation binding constant K₁ of the competitor. The simulation was performed using equation 2 and with competitor concentration of 5 μM (FIG. 1A) and 10 μM (FIG. 1B). The protein and reference compound concentration was 2 and 50 μM, respectively. The ratio of the WaterLOGSY signal for the reference compound in the presence and absence of the competitor is displayed on the Y axis and the dissociation binding constant (in μM) for the competitor is displayed on the X axis. The value 1 on the Y axis corresponds to the signal of the reference compound observed in the absence of the competitor plus the offset arising from the hydration of the free ligand. The value 0 on the Y axis corresponds, in the approximation of only one protein binding site for the reference compound, to the WaterLOGSY signal of the compound in the absence of the protein. Simulations were performed for four different binding constants K_{D} of the reference compound (values indicated on the graph).

FIG. 2. One-Dimensional WaterLOGSY spectra recorded for a 5 μM Human Serum Albumin (HSA) solution in the presence of 50 μM 5-CH₃ D,L Trp (top), 50 μM 6-CH₃ D,L Trp (center) and 50 μM 7-CH₃ D,L Trp (bottom). The displayed expanded spectral region contains the methyl group signals. The spectra were recorded with 2048 scans, 2.6 second (s) repetition time and 1.5 s mixing time. Positive and negative signals identify HSA binding and noninteracting molecules, respectively.

FIG. 3. Isothermal titration calorimetry (ITC) data measured on the binding of tryptophan analogues to HSA. The top panel shows the raw heat data obtained over a series of injections of 7-CH₃ Trp (a), 5-CH₃ D,L Trp (b) and 6-CH₃ D,L Trp (c) into HSA. The integrated heat signals shown in the top panel of the figure gave rise to the normalized binding isotherms shown in the lower panel (7-CH₃ D,L Trp: open circles, 6-CH₃ D,L Trp: solid squares, 5-CH₃ D,L Trp: solid triangles). Dilution heats were collected in blank titrations and were subtracted from the data. No net binding heat effects were observed for 5-CH₃ D,L Trp and 7-CH₃ D,L Trp, respectively indicating that these compounds do not interact with HSA whereas using 6-CH₃ D,L Trp negative binding heats were observed. The solid line represents a calculated curve using the best-fit parameters obtained by a nonlinear least-squares fit to the measured data. The calculated binding parameters were: Stoichiometry (N): 0.98, KB: 2.7±0.2 10⁴ Mol⁻¹, ΔH^{obs}: 1.9±0.1 kcal/mol, ΔS: 13.79 cal/(mol K).

FIG. 4. One-Dimensional WaterLOGSY spectra recorded for 50 μM 6-CH₃ D,L Trp (a), for 50 μM 6-CH₃ D,L Trp with 5 μM HSA in the absence (b) and in the presence (c) of the three compound mixture (10 μM Sucrose, 10 μM 7-CH₃ D,L Trp and 10 μM Diazepam). The displayed spectral region contains the 6-CH₃ signal of the tryptophan derivative. The spectra were acquired with 4096 scans, 2.6 s repetition time and 1.5 s mixing time. The length of the double spin-echo was 25.2 milliseconds (ms) in order to destroy most of the protein signals therefore obtaining a flat baseline.

FIG. 5. One-Dimensional WaterLOGSY spectra recorded for a 5 μM HSA solution with 50 μM 6-CH₃ D,L Trp in the absence (a) and in the presence of 10 μM sucrose (b), 10 μM 7-CH₃ D,L Trp (c) and 10 μM Diazepam (d). The displayed spectral region contains the 6-CH₃ signal of the tryptophan derivative. The other experimental conditions are the same as in FIG. 4.

### DETAILED DESCRIPTION OF ILLUSTRATIVE EMBODIMENTS OF THE INVENTION

The present invention is directed to the use of WaterLOGSY in competition binding experiments. Competition binding experiments involve the displacement of a reference compound in the presence of a competing molecule. Preferably, the reference compound interacts with the target molecule with a binding affinity in the micromolar range. Preferably, the test compound interacts with the target molecule with a binding affinity stronger than (i.e., less than) 1 micromolar (e.g., in the nanomolar range).

Although this method is particularly useful for identifying ligands that are relatively strong binders to the target molecule, it can be used for identifying ligands of a wide range of binding affinities. The relatively strong binders are typically defined as those having a dissociation binding constant K_{D} of less than about 1 micromolar, preferably less than about 500 nM, more preferably less than about 100 nanomolar (nM).

The WaterLOGSY method (also referred to as the Water-Ligand Observed via Gradient Spectroscopy Y) is based on the transfer of magnetization from the protons of bulk water to the protons of compounds that interact with target molecules (e.g., proteins). Using WaterLOGSY techniques, binding compounds are distinguished from nonbinders by the opposite sign of their water-ligand nuclear Overhauser effects (NOEs). The WaterLOGSY method is described in greater detail in International Publication No. WO 01/23330 (published Apr. 5, 2001) and in C. Dalvit et al., J. Biomol. NMR, 18, 65-68 (2000).

This description focuses on proteins as the target molecules, although it applies also to other macromolecules that can be considered “target molecules” (e.g., DNA, RNA). More specifically, this NMR experiment utilizes the large bulk water magnetization to transfer magnetization via the protein-ligand complex to the free ligand (or potential ligand) in a selective manner. In this experiment, the proton resonances of non-interacting compounds appear with opposite sign and tend to be weaker than those of the interacting ligands.

The WaterLOGSY method is based on the fact that water molecules link the ligand to the protein, with most of the water molecules making three or more hydrogen bonds. In addition to these bridging water molecules, other water molecules are identified at the binding site. Selective excitation of the protons of the water molecules followed by a mixing time effectively transfers magnetization from the bulk water to the protein-ligand complex with the same sign as the starting magnetization. That is, this method involves the transfer of magnetization from bound water to nearby protons of the compounds that interact with the protein in a “protein-ligand” complex. The magnetization transfer from water to the protein-ligand complex can be supplemented by chemical exchange with the protons of labile functional groups. Both processes act constructively to transfer magnetization from the bulk water to the protein-ligand complex.

Typically, 1D WaterLOGSY experiments are performed by either selective decoupling or inversion of the water signal. Non-interacting compounds are characterized by negative intensity in WaterLOGSY spectra, while compounds that interact with the protein are characterized by positive intensity.

The pulse sequence of the WaterLOGSY method typically involves a first element of a 90° nonselective RF pulse, a 180° selective RF pulse, and a 90° nonselective RF pulse, followed by a second element of a specific mixing time (typically 1-2 seconds) for magnetization transfer, followed by a third element of signal detection. The first element can also simply involve a single 180° selective RF pulse. Other pulse sequences can also be used in the first element as long as the water is selectively excited. A “selective” pulse is one that is ideally tuned to a specific frequency that is matched to a specific nuclear spin (i.e., it is specific for a particular proton), in this case, the proton nuclei of water. A “nonselective” pulse is one that is not tuned to a specific frequency, but excites wide range of frequencies. The third element of signal detection can involve additional radio frequency (RF) pulses to reduce the water signal. Examplary pulse sequences for suppressing the water signal are disclosed, for example, in W. S. Price, Annual Reports on NMR Spectroscopy, 1999, 38, 289-354.

The WaterLOGSY method involves generating a ¹H NMR spectrum of one or more compounds, adding a target molecule, and generating a ¹H NMR spectrum of the mixture. Typically, WaterLOGSY experiments involve the use of 1D NMR, although 2D NMR experiments can be run. Such 2D experiments involve 2D homonuclear ¹H/¹H experiments, which are well known to one of skill in the art.

WaterLOGSY represents a powerful method for primary NMR screening in the identification of compounds interacting with macromolecules, including proteins and DNA or RNA fragments. For example, the method is useful for the detection of compounds binding to a receptor with a binding affinity in the μM range. The method is somewhat limited, however, as with all the techniques that detect ligand resonances, in its ability to detect strongly binding ligands (i.e., those having a slow dissociation rate). The present invention overcomes this problem through the use of a reference compound with a known K_{D} in the μM range together with properly-designed competition binding experiments (c-WaterLOGSY), which permits the detection of strong binders.

Generally, the method of the present invention includes: providing a reference compound that interacts with the target molecule; collecting a WaterLOGSY nuclear magnetic resonance spectrum of the reference compound in the presence of the target molecule; providing a test sample comprising at least one test compound; collecting a WaterLOGSY nuclear magnetic resonance spectrum of the reference compound in the presence of the test sample and the target molecule; and comparing the WaterLOGSY spectra to determine if at least one test compound interacts with the target molecule by displacing the reference compound. Preferably, the test compound has a binding affinity to the target molecule tighter than that of the reference compound.

As shown in the Experimental Section, a mathematical expression can be used to determine the appropriate NMR experimental conditions and for an approximate determination of the binding constant (i.e., dissociation constant).

For the optional experimental determination of the dissociation constant of a ligand, the method further includes the following steps: collecting a WaterLOGSY nuclear magnetic resonance spectrum of the reference compound in the absence of the target molecule; and comparing the WaterLOGSY spectra of the reference compound in the presence of the target molecule, and in the absence of the target molecule, and in the presence of the test sample and target molecule.

As discussed above, the step of comparing the WaterLOGSY spectra to determine if at least one test compound interacts with the target molecule involves evaluating at least one reference compound resonance for a change in sign (i.e., by virtue of the opposite sign of their water-ligand nuclear Overhauser effects (NOEs)). Alternatively, however, this can involve evaluating at least one reference compound resonance for a reduction in signal intensity (i.e., by virtue of a decreased fraction of bound reference compound).

The reference compound can be identified as well using WaterLOGSY, as well as other methods such as spectroscopic or biochemical assays, which are well known to one of skill in the art. Preferably, the reference compound can be identified by the following steps: collecting a WaterLOGSY nuclear magnetic resonance spectrum of a potential reference compound in the absence of the target molecule; collecting a WaterLOGSY nuclear magnetic resonance spectrum of the potential reference compound in the presence of the target molecule; and comparing the WaterLOGSY spectra to identify whether the potential reference compound interacts with the target molecule.

Optionally, prior to collecting a WaterLOGSY nuclear magnetic resonance spectrum of a reference compound in the presence of a target molecule for use in the comparing step, the method includes: collecting WaterLOGSY nuclear magnetic resonance spectra of the reference compound in the presence of the target molecule at different concentrations of the target molecule or at different concentrations of the reference compound; and determining the optimum experimental conditions for identifying at least one test compound that interacts with the target molecule.

The target molecules that can be used in the methods of the present invention include a wide variety of molecules, particularly macromolecules, such as polypeptides (preferably, proteins), polynucleotides, organic polymers, and the like.

“Polynucleotide” as used herein refers to a polymeric form of nucleotides of any length, either ribonucleotides or deoxynucleotides, and includes both double- and single-stranded DNA and RNA. A polynucleotide may include both coding and non-coding regions, and can be obtained directly from a natural source (e.g., a microbe), or can be prepared with the aid of recombinant, enzymatic, or chemical techniques. A polynucleotide can be linear or circular in topology. A polynucleotide can be, for example, a portion of a vector, such as an expression or cloning vector, or a fragment.

“Polypeptide” as used herein refers to a polymer of amino acids and does not refer to a specific length of a polymer of amino acids. Thus, for example, the terms peptide, oligopeptide, protein, and enzyme are included within the definition of polypeptide. This term also includes post-expression modifications of the polypeptide, for example, glycosylations, acetylations, phosphorylations, and the like.

The reference compound is one that interacts with the selected target molecule with a binding affinity sufficiently low that it gives rise to a readily observed, positive-intensity WaterLOGSY signal in the presence of the target molecule. Preferably, a weakly binding reference compound is used. Relatively weakly binding reference compounds are typically defined as those having a dissociation binding constant K_{D} of at least about 10 micromolar or higher.

The reference compound preferably includes methyl groups, which typically provide a strong WaterLOGSY signal. Such methyl groups often are less hydrated, resulting in a smaller WaterLOGSY signal for these reference compounds when free in solution.

The test compounds that can be evaluated can be any of a wide variety of compounds, which potentially have a wide variety of binding affinities to the target. Significantly, the method of the present invention has the ability to detect compounds that are relatively strong binders. The relatively strong binders are typically defined as those having a dissociation binding constant K_{D} of less than about 1 micromolar. Compounds that can be screened using the method of the present invention include, for example, plant extracts, fungi extracts, other natural products, and libraries of small organic molecules.

The present invention can screen for ligands from a library of compounds that have a broad range of solubilities (the methods are particularly amendable to compounds having very low solubilities). Significantly and advantageously, for certain embodiments, the present invention preferably involves carrying out a binding assay at relatively low concentrations of target (i.e., target molecule) and low ratios of test compound to target. Thus, preferred embodiments of the present invention allow for the detection of compounds that are only marginally soluble. Typically and preferably, the test compound has a solubility in water of no greater than about 10 μM.

Preferably, the concentration of each test compound in each sample is no greater than about 100 μM, although higher concentrations can be used if desired. However, a significant advantage of the method of the present invention is that very low ligand concentrations (e.g., no greater than about 10 μM) can be used. Preferably, the concentration of target molecule is about 1 μM to about 10 μM.

The exact concentrations and ratios of test compound to target molecule used can vary depending on the size of the target molecule, the amount of target molecule available, the desired binding affinity detection limit, and the desired speed of data collection. Although it is desirable to use the method of the present invention to detect strongly binding ligands, those that are moderately and even weekly binding can be detected if desired. As described in greater detail below, the lower limit in affinity strength for the detection can be tuned by properly selecting the reference compound (i.e., different K_{D}) and/or different [I_{TOT}]/[L_{TOT}] ratios according to equation (2).

The present invention could also find useful applications for rapid screening of chemical mixtures (i.e., mixtures of two or more test compounds). Rapid screening techniques typically involve providing a plurality of test samples, each test sample comprising one or more test compound (and often a mixture of two or more test compounds).

Once a ligand (preferably a high affinity ligand) has been identified and confirmed, its structure is used to identify available compounds with similar structures to be assayed for activity or affinity, or to direct the synthesis of structurally related compounds to be assayed for activity or affinity. These compounds are then either obtained from inventory or synthesized. Most often, they are then assayed for activity using enzyme assays. In the case of molecular targets that are not enzymes or that do not have an enzyme assay available, these compounds can be assayed for affinity using NMR techniques similar to those described above, or by other physical methods such as isothermal denaturation calorimetry.

In some instances, ligand binding is further studied using more complex NMR experiments or other physical methods such as calorimetry or X-ray crystallography.

### EXAMPLES

Objects and advantages of this invention are further illustrated by the following examples, but the particular materials and amounts thereof recited in these examples, as well as other conditions and details, should not be construed to unduly limit this invention.

Experimental Protocol

Fatty acid free human serum albumin (A-3782) was purchased from Sigma and used without further purification. Sucrose (S7903) and 7-CH₃-D,L-Trp (M8379) were purchased from Sigma, 5-CH₃-D,L-Trp (69560) and 6-CH₃-D,L-Trp (69570) were purchased from Fluka. Diazepam was purchased from Carlo Erba. The NMR samples were in phosphate buffered saline (PBS) buffer (Sigma) pH 7.4. D₂O was added to the solutions (8% final concentration) for the lock signal.

NMR Experiments. All spectra were recorded at 293 K with a Varian Inova 600 MHz NMR spectrometer equipped with a 5 millimeter (mm) triple-resonance inverse probe and an autosampler. For each sample a reference spectrum and a ID WaterLOGSY spectrum were recorded. The details of the pulse sequence version used for the WaterLOGSY experiment reported here can be found in the literature (C. Dalvit et al., J. Biomol. NMR, 21, 349-359 (2001)). The 1^{st} water selective 180° pulse was 25 milliseconds (ms) long. A weak rectangular PFG is applied during the entire length of the mixing time (1.5 seconds (s)). A short gradient recovery time of 2 ms was introduced at the end of the mixing time before the detection pulse. The two water selective 180° square pulses of the double spin-echo scheme (T-L. Hwang et al., J. Magn. Reson. A 1995, 112, 275-279) were 2.6 ms long. The gradient recovery time was 0.2 s. The data were collected with a sweep width of 7407 Hz, an acquisition time of 0.648 s, and a relaxation delay of 2.648 s. Prior to Fourier transformation the data were multiplied with an exponential function with a line broadening of 1 Hz.

ITC Experiments. Calorimetric measurements were carried out at 298 K in PBS buffer (Sigma) at a protein concentration of 30 μM and a ligand concentration of 1.5 mM using a VP-ITC titration calorimeter (MicroCal). Heats of dilution were measured in blank titrations by injecting the protein into the buffer used in the particular experiment and were subtracted from the binding heats. Thermodynamic parameters were determined by non-linear least squares methods using routines included in the Origin software package (MicroCal).

Results and Discussion

NMR competition binding experiments performed with known inhibitors have been used in order to determine the specificity of the identified NMR-hits. These competition binding WaterLOGSY experiments, properly designed, can be used to screen chemical mixtures for the detection of strong ligands to the protein of interest. These experiments are referred to as c (Competition)-WaterLOGSY experiments.

The intensity of the experimental WaterLOGSY signal I_{WL} for a proton i of a reference compound is provided by the expression:

$${I}_{WL} - [ L ] ( {\sum }_{j} {σ}_{i j}^{free} + {\sum }_{w} {σ}_{i w}^{free} ) ∝ [ EL ] ( {\sum }_{j} {σ}_{i j}^{bound} + {\sum }_{k} {σ}_{i k} + {\sum }_{w} {σ}_{i w}^{bound} ) \qquad ( 1 )$$

The σ are the different cross relaxation rates involving the proton i in the bound and free state, respectively. The indices j are ligand exchangeable protons, k are protein protons near ligand and w are water molecules near ligand. The quantities [L] and [EL] correspond to the concentration of free and bound ligand, respectively. The two concentrations are related to each other via the equation [L]=[L_{TOT}]−[EL] where [L_{TOT}] is the total ligand concentration. If [L_{TOT}]>>[E_{TOT}] (total protein concentration), [L_{TOT}] can replace [L] in equation (1). The term

$$[ {L}_{TOT} ] ( {\sum }_{j} {σ}_{i j}^{free} + {\sum }_{w} {σ}_{i w}^{free} )$$

corresponds to the experimental hydration of the proton i of the reference compound in the absence of the protein. Herein, the term I_{WLOGSY} refers to the intensity of the measured WaterLOGSY signal I_{WL} plus the correction term obtained from an experiment recorded for the ligand in the absence of the protein.

In the presence of a competitive molecule the protein bound concentration of the reference compound diminishes (A. Fersht, Enzyme Structure and Mechanism W. H. Freeman and Company New York 1985, pages 98-120. The WaterLOGSY signal intensity ratio for a reference compound in the presence and absence of a competitor is given by the equation (2):

$$\frac{{I}_{WLOGSY} ( + )}{{I}_{WLOGSY} ( - )} = \frac{\begin{matrix}[ {E}_{TOT} ] + [ {L}_{TOT} ] + {K}_{D} ( 1 + \frac{[ I ]}{{K}_{I}} ) - \\ \sqrt{{\{ [ {E}_{TOT} ] + [ {L}_{TOT} ] + {K}_{D} ( 1 + \frac{[ I ]}{{K}_{I}} ) \}}^{2} - 4 [ {E}_{TOT} ] [ {L}_{TOT} ]}\end{matrix}}{\begin{matrix}[ {E}_{TOT} ] + [ {L}_{TOT} ] + {K}_{D} - \\ \sqrt{{\{ [ {E}_{TOT} ] + [ {L}_{TOT} ] + {K}_{D} \}}^{2} - 4 [ {E}_{TOT} ] [ {L}_{TOT} ]}\end{matrix}}$$

where I_{WLOGSY}(+) and I_{WLOGSY}(−) are the intensity of the reference compound in the presence and absence of the competitor, respectively. The quantities [E_{TOT}], [L_{TOT}] and [I_{TOT}] are the protein, reference compound and competitor concentration, respectively. The quantities K_{D} and K₁ are the dissociation binding constants for the reference compound and the competitor, respectively. In deriving equation (2), the absence of positive or negative cooperativity effects was assumed.

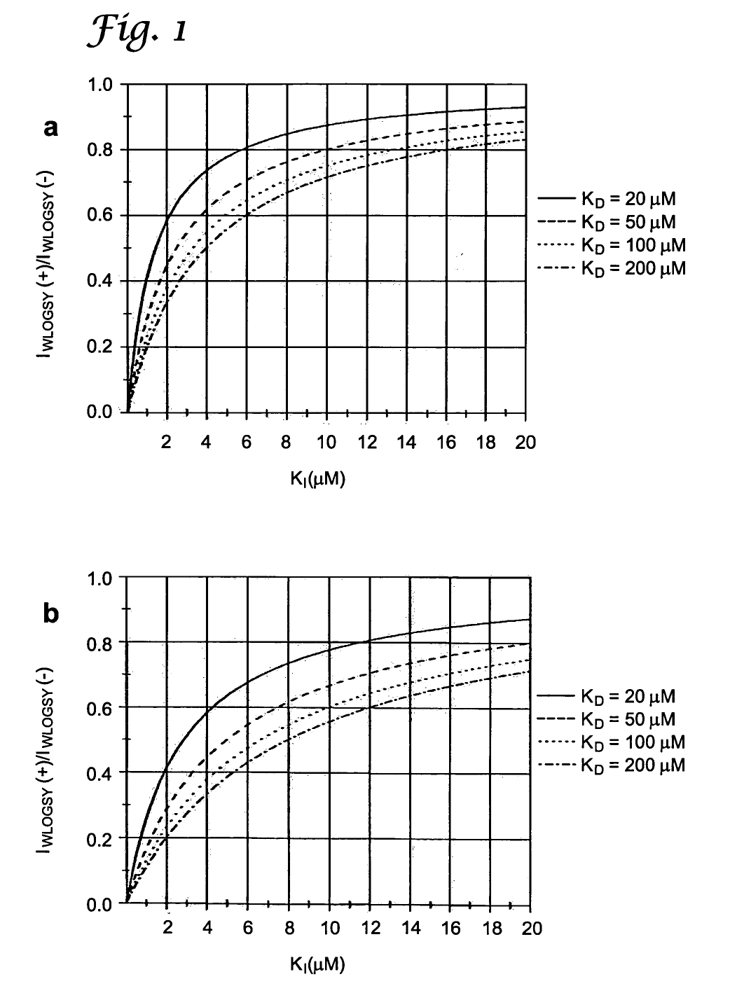

FIG. 1 shows a simulation of the WaterLOGSY signal of the reference compound as a function of the K₁, of a competitor. For the simulation, a reference compound and protein concentration of 50 μM and 2 μM, respectively, were assumed. Two different concentrations, 5 μM and 10 μM, have been considered for the competitor.

From this simulation it is evident that the signal attenuation of the reference compound in the presence of a competitor depends upon K_{D}, K₁ and [I_{TOT}]. Therefore it is possible to detect indirectly the presence of a strong inhibitor in a chemical mixture simply by monitoring the WaterLOGSY signal of a reference compound. The lower limit in affinity strength for the detection can be tuned by properly selecting the reference compound (i.e., different K_{D}) and/or different [I_{TOT}]/[L_{TOT}] ratios according to equation (2).

This approach requires first the identification with NMR or other techniques of a weak affinity ligand to the protein target of interest. The binding constant for this compound should be calculated in order to properly design the experiments according to equation (2) and FIG. 1. When possible a compound with a methyl group should be chosen in order to maximize the sensitivity of the experiment. This will allow reduction in protein consumption.

A well characterized protein, Human Serum Albumin (HSA) was chosen as a test case for demonstrating the application of the c-WaterLOGSY. Drugs such as for example naproxen, diazepam and ibuprofen are known to bind to HSA on site II (Peters Theodore Jr., All about Albumin Biochemistry, Genetics, and Medical Applications Academic Press, San Diego, U.S.A. 1996, pages 109-116. The endogenous aminoacid tryptophan binds also on site II of HSA (McMenamy, R. H.; Oncley, J. L. The specific binding of L-tryptophan to serum albumin. J Biol. Chem. 1958, 233, 1436-1447).

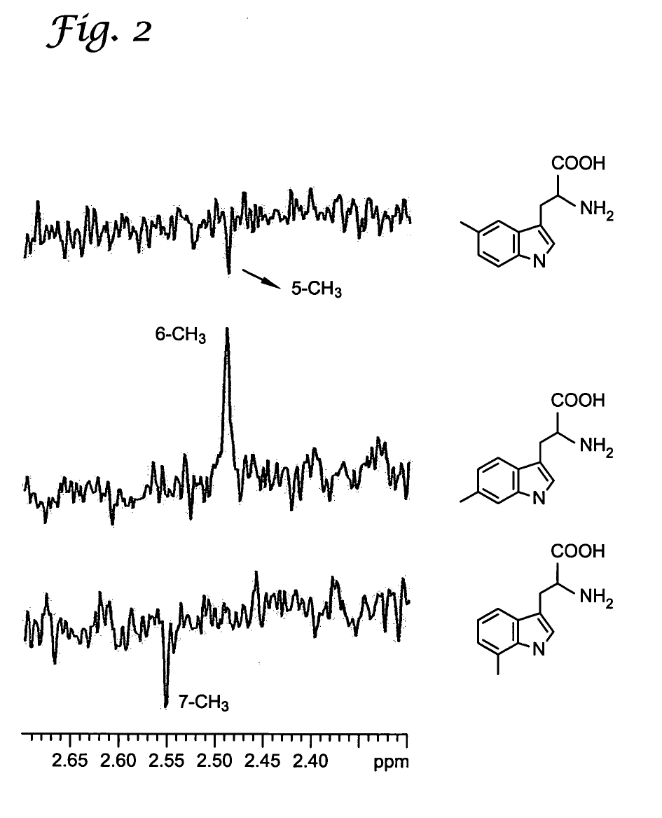

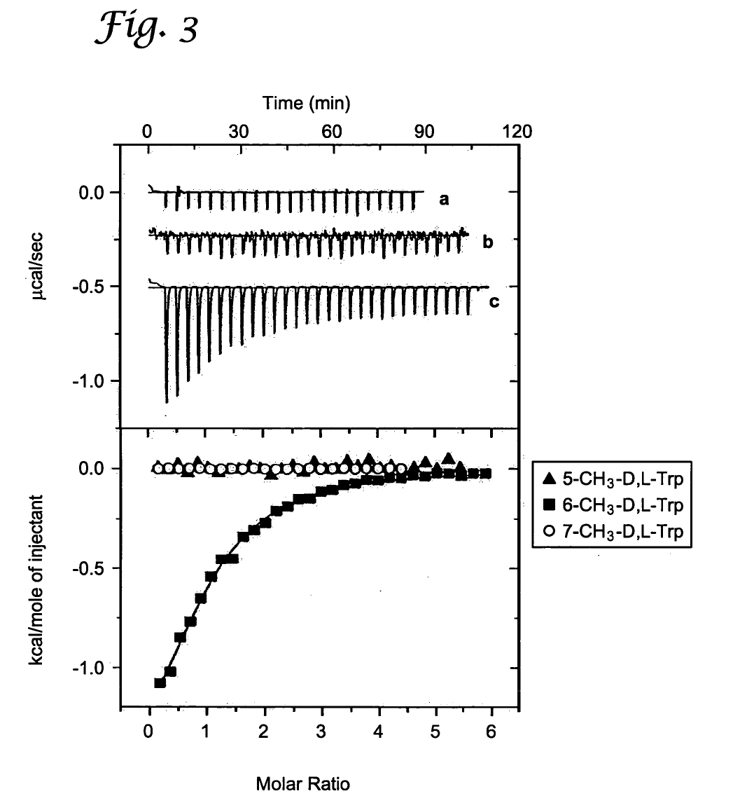

Therefore in an effort to identify a potential reference molecule, three methyl-tryptophan derivatives, namely 5-CH₃, 6-CH₃, and 7-CH₃ Trp, were selected. The spectra of the three derivatives, shown in FIG. 2, identified the 6-CH₃ Trp as a ligand for HSA. The other two derivatives 5-CH₃ and 7-CH₃ Trp do not interact with the protein as indicated by the negative signals in FIG. 2. The simple substitution of the proton with a methyl group at position 5 or 7 on the ring abolishes completely the binding to HSA. These findings were confirmed by ITC measurements as shown in FIG. 3. In addition this techniques provided an association binding constant (K_{B}) for the selected reference compound (i.e. 6-CH₃ Trp) which was determined to be 2.7±0.2 10⁴ Mol⁻¹.

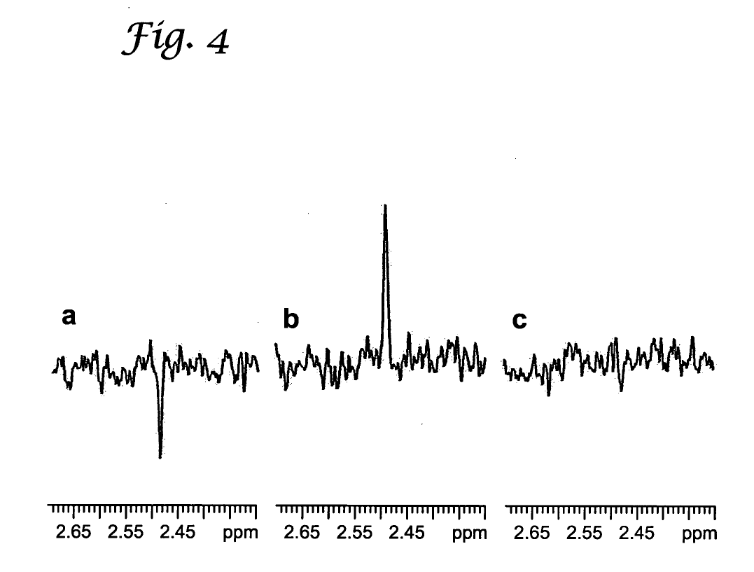

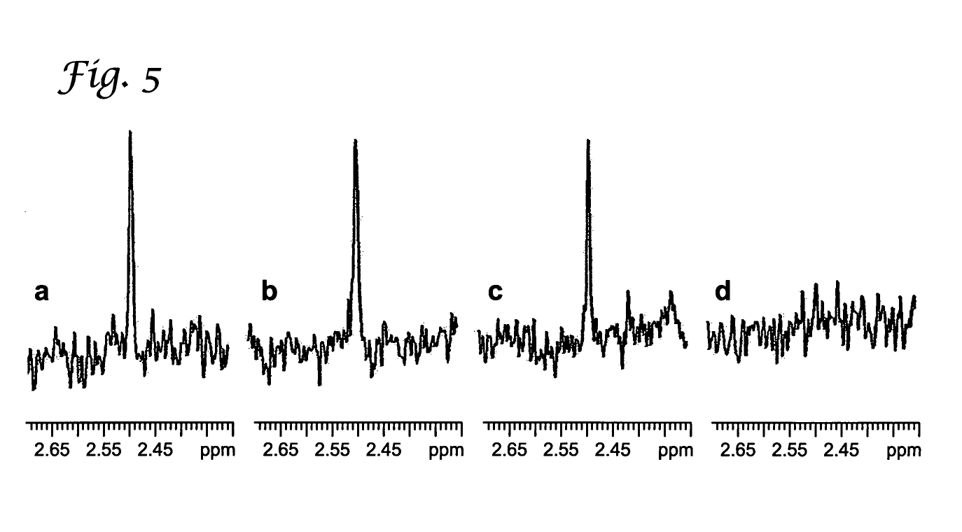

In the c-WaterLOGSY approach a spectrum is first acquired for the selected reference compound in the absence of the protein. This allows for extracting the hydration correction term discussed above. Then, an identical spectrum is acquired for the reference compound in the presence of the protein. These two spectra are acquired only once and are then used for the analysis of all the screened chemical mixtures. A small spectral region containing the methyl group of 6-CH₃ Trp in the absence and presence of HSA is shown in FIG. 4a,b, respectively. Subsequently, WaterLOGSY spectra are acquired for compound mixtures (Sucrose, 7-CH₃ Trp and Diazepam in this example) in the presence of the protein and the reference compound as shown in FIG. 4c. The WaterLOGSY signals of the reference compound in the absence and presence of the mixture are then compared. A change in sign or substantial signal reduction (as shown in FIG. 4c) of the reference compound resonance in the spectrum recorded in the presence of the mixture is an indication that one or more compounds comprising the mixture is a potent ligand and displaces the reference compound (6-CH₃ Trp) from the protein. Deconvolution of the chemical mixture performed in the presence of the reference compound is shown in FIG. 5. No signal intensity change of 6-CH₃ Trp was observed in the presence of sucrose (FIG. 5b) and 7-CH₃ Trp (FIG. 5c) whereas a drastic signal reduction was observed in the presence of Diazepam (FIG. 5d). This deconvolution enables the identification of Diazepam as the high affinity ligand present in the mixture.

The calculation of the signal reduction (with the proper correction) and the knowledge of K_{D} of the reference compound (6-CH₃ Trp) provide an approximate estimation or a lower limit, according to equation (2), of the dissociation binding constant of the identified ligand (Diazepam). This is achieved with a single point measurement since equation (2) considers also the effect deriving from the protein concentration term. This contribution is neglected in the IC₅₀ equation derived in the literature (Y.-C. Cheng et al., Biochem. Pharmacology 1973, 22, 3099-3108) and used in NMR studies (M. Mayer et al., J. Am. Chem. Soc. 2001, 123, 6108-6117) where the concentration of both ligands is considered much larger compared to the protein concentration and K_{D}.

Equation (2) is a general expression and should be applicable to other NMR parameters investigated in competition binding experiments. With a signal reduction of 65% and a K_{D} of 37 μM for 6-CH₃ Trp, a binding constant for diazepam of 2 μM+/−1 μM was estimated, which is close to the value of 2.6 μM reported in the literature (U. Kragh-Hansen, Biochem. J. 1991, 273, 641-644). Note that with equation (2) it is possible to measure very strong binding ligands with binding constants in the nM range. For this purpose it is necessary to use even a lower competitive inhibitor concentration (nM).

The procedure described here can also be applied to the identification of high affinity ligands present in plant or fungi extracts. The composition and concentration of the different components present in the extracts is not known. Nevertheless, the knowledge of the presence of a strong binding ligand in the extract can guide the chemist in the separation and isolation of the active compound.

It is recommended that a weakly binding reference compound be used in all the WaterLOGSY experiments. In the search of weak and medium strength inhibitors the concentration of the mixture constituents should be the same as for the reference compound (e.g., K_{D} of 50 μM). The characteristic appearence of the positive signals for a compound of the mixture will identify that molecule as a ligand to the target of interest. If this is associated with no signal reduction for the reference compound it is possible to conclude, according to equation (2), that the compound does not compete with the reference molecule and binds on a different site of the protein. However, the absence or strong reduction of the positive signals for the reference compound is an indication that one of the molecules comprising the mixture is an high affinity ligand. Deconvolution of the mixture will then allow the identification of the molecule. Using this approach both weak and strong inhibitors will be detected. The c-WaterLOGSY technique was successfully applied in the search of strong kinase inhibitors that bind in the ATP binding site. Protein and ligand concentration as low as 2 μM and 5 μM, respectively, were employed in these studies therefore allowing the identification of strong inhibitors that are only marginally soluble (data not shown).

Conclusion

This data has shown that the use of a medium-low affinity reference compound together with properly designed c-WaterLOGSY experiments permit the indirect detection of high affinity ligands. An approximate value or a lower limit of the dissociation binding constant of the identified molecule can be extracted with a single point measurement. The technique is particularly suitable for rapid screening of chemical mixtures and natural product extracts. Finally, the experiment is not limited to the interactions of small molecules with proteins, but can be used efficiently also in the identification of molecules interacting with DNA or RNA fragments.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein. Such examples and embodiments are presented by way of example only with the scope of the invention intended to be limited only by the claims set forth herein as follows.

What is claimed is:

1. A method of identifying a ligand to a target molecule, the method comprising:
providing a reference compound that interacts with the target molecule;
collecting a first WaterLOGSY nuclear magnetic resonance spectrum of the reference compound in the presence of the target molecule;
providing a test sample comprising at least one test compound;
collecting a second WaterLOGSY nuclear magnetic resonance spectrum of the reference compound in the presence of the test sample and the target molecule; and
comparing the first and second WaterLOGSY spectra to determine if at least one test compound interacts with the target molecule by displacing the reference compound.

2. The method of claim 1 wherein the test compound has a binding affinity to the target molecule tighter than that of the reference compound.

3. The method of claim 1 further comprising:
collecting a third WaterLOGSY nuclear magnetic resonance spectrum of the reference compound in the absence of the target molecule; and
comparing the first, second, and third WaterLOGSY spectra to determine the dissociation constant of the test compound.

4. The method of claim 1 wherein the target molecule is a macromolecule.

5. The method of claim 4 wherein the macromolecule is a polypeptide or a polynucleotide.

6. The method of claim 4 wherein the macromolecule is a protein.

7. The method of claim 1 wherein the test compound has a solubility in water of no greater than about 10 micromolar.

8. The method of claim 1 wherein the reference compound interacts with the target molecule with a binding affinity in the micromolar range.

9. The method of claim 1 wherein the test compound interacts with the target molecule with a binding affinity stronger than 1 micromolar.

10. The method of claim 1 wherein comparing the WaterLOGSY spectra to determine if at least one test compound interacts with the target molecule comprises evaluating at least one reference compound resonance for a change in sign.

11. The method of claim 1 wherein comparing the WaterLOGSY spectra to determine if at least one test compound interacts with the target molecule comprises evaluating at least one reference compound resonance for a reduction in signal intensity.

12. The method of claim 1 further comprising a step of identifying the reference compound comprising:
collecting a WaterLOGSY nuclear magnetic resonance spectrum of a potential reference compound in the absence of the target molecule;
collecting a WaterLOGSY nuclear magnetic resonance spectrum of the potential reference compound in the presence of the target molecule; and
comparing the WaterLOGSY spectra of the potential reference compound in the presence and the absence of the target molecule to identify whether the potential reference compound interacts with the target molecule.

13. The method of claim 1 wherein the reference compound comprises a methyl group.

14. The method of claim 1 wherein the test sample comprises a mixture of two or more test compounds.

15. The method of claim 1 wherein providing a test sample comprises providing a plurality of test samples, each test sample comprising one or more test compounds.

16. The method of claim 15 wherein each test sample comprises a mixture of two or more test compounds.

17. The method of claim 1 wherein prior to collecting a WaterLOGSY nuclear magnetic resonance spectrum of the reference compound in the presence of the target molecule for use in the comparing step, the method comprises:
collecting WaterLOGSY nuclear magnetic resonance spectra of the reference compound in the presence of the target molecule at different concentrations of the target molecule or at different concentrations of the reference compound; and
determining the optimum experimental conditions for identifying at least one test compound that interacts with the target molecule.
